(19) United States
(12) Patent Application Publication
(10) Pub. No.: US 2023/0200742 A1
(43) Pub. Date: Jun. 29, 2023

(54) METHOD AND APPARATUS FOR CLASSIFYING HEARTBEATS AND METHOD OF TRAINING HEARTBEAT CLASSIFICATION MODEL

(71) Applicant: ELECTRONICS AND TELECOMMUNICATIONS RESEARCH INSTITUTE, Daejeon (KR)

(72) Inventors: MUHAMMAD ZUBAIR, Daejeon (KR); Sung Pil WOO, Daejeon (KR); Chan Won PARK, Daejeon (KR); Sun Hwan LIM, Daejeon (KR)

(21) Appl. No.: 18/083,935

(22) Filed: Dec. 19, 2022

(30) Foreign Application Priority Data
Dec. 23, 2021 (KR) 10-2021-0185838

(51) Int. Cl. A61B 5/00; A61B 5/318

(52) U.S. Cl. CPC A61B 5/7267; A61B 5/318

(57) ABSTRACT

A computing device inputs a sample generated from an electrocardiogram signal to a heartbeat classification model, generates a feature map from the sample through multiple first layers of the heartbeat classification model, generates an attention mask based on an assistant feature generated from the feature map and the sample, generates a masked feature map by masking the feature map with the attention mask; and performs classification of the sample from the masked feature map through a second layer of the heartbeat classification model.

### CROSS-REFERENCE TO RELATED APPLICATION(S)

The present application claims priority under 35 U.S.C. §119(a) to Korean Application No. 10-2021-0185838, filed on Dec. 23, 2021 in the Korean Intellectual Property Office, which is hereby incorporated by reference for all purposes as if set forth herein.

### BACKGROUND

### 1. Technical Field

Embodiments of the present invention relate to a method and apparatus for classifying heartbeats and a method of training a heartbeat classification model.

### 2. Description of Related Art

Automatic classification of electrocardiograms (ECGs) is a very difficult task and is still regarded as an open problem for several reasons, despite efforts of researchers. For example, morphological characteristics of an ECG waveform vary from patient to patient and may be modulated by ongoing physiological processes and patient’s physiological states. Thus, a classification system designed for a certain group of patients may not operate well on data obtained from other patients. In this regard, numerous deep learning-based heartbeat classification methods have been proposed to achieve a better generalization function by addressing problems related to existing machine learning methods. However, deep representation learning of heartbeats is still difficult. For example, quality of deep features is closely related to the content of information processed by deep models.

Another problem is class imbalanced data, which can cause a skewed distribution of data. In the imbalanced data, some classes (minority classes) provide insufficient representation whereas other classes provide very rich representation. High representation of a majority class during training can bias the classification algorithm towards the majority class. High representation of the majority class during training can distort a classification algorithm to the majority class. As a result, minority class samples may often not be classified. However, in actual scenarios, infrequent events can be very important. For example, in surveillance operation, suspicious activity is a rare event that a monitoring system is required to correctly recognize. Similarly, disease diagnosis in medicine is an example of a scenario where the minority class samples become main concerns. In particular, since arrhythmias are sudden and rare, the task of detecting arrhythmias is one example of such a scenario. In arrhythmias, detection of abnormal heartbeats is the most important task and misclassification of the abnormal heartbeats is undesirable.

### <Related Literature>

(Patent Document 1) KR 10-2171236 B1

### SUMMARY

Embodiments of the present invention provide a method and apparatus for classifying heartbeats and a method of training a heartbeat classification model, which can extract features having high relevancy or can solve a problem of unbalanced data.

In accordance with one aspect of the present invention, there is a heartbeat classification method carried out by a computing device. The heartbeat classification method includes the steps of: inputting a sample generated from an electrocardiogram signal to a heartbeat classification model; generating a feature map from the sample through multiple first layers of the heartbeat classification model; generating an attention mask based on an assistant feature generated from the feature map and the sample; generating a masked feature map by masking the feature map with the attention mask; and performing classification of the sample from the masked feature map through a second layer of the heartbeat classification model.

In some embodiments, the assistant feature may include an RR interval of the electrocardiogram signal corresponding to the sample.

In some embodiments, the step of generating an attention mask may include: calculating a statistical value of the feature map; generating a normalized statistical value through normalization of the statistical value with the assistant feature; and generating the attention mask from the normalized statistical value through at least one activation layer.

In some embodiments, the step of generating an attention mask may further include inputting the normalized statistical value to the activation layer after multiplying the normalized statistical value by a weight value and shifting the normalized statistical value by a predetermined value.

In some embodiments, the multiple first layers may include multiple convolution layers and the second layer may include at least one fully-connected layer.

In accordance with another aspect of the present invention, a method of training a heartbeat classification model carried out by a computing device may be provided. The method includes the steps of:, upon classification of multiple samples generated from an electrocardiogram signal into multiple class clusters according to classes to which labels of the samples pertain, selecting a first sample from a majority class cluster having the largest number of samples among the multiple class clusters based on a predetermined criterion; converting the first sample into a second sample pertaining a source class cluster having the smallest number of samples among the multiple class clusters; generating a data set from the multiple samples and the second sample; and training the heartbeat classification model by inputting an input sample selected from the data set to the heartbeat classification model.

In some embodiments, the predetermined criterion may include similarity to a sample pertaining to the source class cluster.

In some embodiments, the step of selecting a first sample may include: calculating a first distance from the first sample to a sample corresponding to a center of the source class cluster; calculating a second distance from the first sample to a sample corresponding to a center of each of remaining class clusters excluding the source class cluster among the multiple class clusters; and selecting the first sample when the first distance is smaller than the second distance.

In some embodiments, the step of converting the first sample into the second sample may include tagging the second sample with a label of the source class cluster.

In some embodiments, the step of converting the first sample into the second sample may include increasing margins between the second sample and boundaries of the remaining class clusters based on a constant factor.

In some embodiments, the step of training the heartbeat classification model may include: generating a feature map from the input sample through multiple first layers of the heartbeat classification model; generating an attention mask based on an assistant feature generated from the feature map and the input sample; generating a masked feature map by masking the feature map with the attention mask; and performing classification of the input sample from the masked feature map through a second layer of the heartbeat classification model.

In some embodiments, the assistant feature may include an RR interval of the electrocardiogram signal corresponding to the input sample.

In some embodiments, the step of generating an attention mask may include: calculating a statistical value of the feature map; generating a normalized statistical value through normalization of the statistical value with the assistant feature; and generating the attention mask from the normalized statistical value through at least one activation layer.

In some embodiments, the step of generating an attention mask may further include inputting the normalized statistical value to the activation layer after multiplying the normalized statistical value by a weight value and shifting the normalized statistical value by a predetermined value.

In some embodiments, the multiple first layers may include multiple convolution layers and the second layer may include at least one fully-connected layer.

In accordance with a further aspect of the present invention, a heartbeat classification apparatus including: a memory storing at least one instruction and a processor may be provided. The processor may execute the instruction to input a sample generated from an electrocardiogram signal to a heartbeat classification model, to generate a feature map from the sample through multiple first layers of the heartbeat classification model, to generate an attention mask based on an assistant feature generated from the feature map and the sample, to generate a masked feature map by masking the feature map with the attention mask; and to perform classification of the sample from the masked feature map through a second layer of the heartbeat classification model.

### DRAWINGS

FIG. 1 is a diagram of one example of a heartbeat classification apparatus according to one embodiment of the present invention.

FIG. 2 is a diagram of one example of a preprocessing unit of the heartbeat classification apparatus according to the embodiment of the present invention.

FIG. 3 is a diagram of one example of a classifier of the heartbeat classification apparatus according to the embodiment of the present invention.

FIG. 4 is a flowchart illustrating one example of a heartbeat classification method or a training method of the heartbeat classification apparatus according to the embodiment of the present invention.

FIG. 5 is a diagram of one example of an attention module of the heartbeat classification apparatus according to the embodiment of the present invention.

FIG. 6 is a diagram of one example of a resampling unit of the heartbeat classification apparatus according to the embodiment of the present invention.

FIG. 7 is a view illustrating a sample suitable for conversion in the heartbeat classification apparatus according to the embodiment of the present invention.

FIG. 8 is a view illustrating conversion of a sample in the heartbeat classification apparatus according to the embodiment of the present invention.

FIG. 9 is a block diagram of one example of a computing device according to one embodiment of the present invention.

### DETAILED DESCRIPTION

Hereinafter, embodiments of the present invention will be described with reference to the accompanying drawings such that the present invention can be easily implemented by those skilled in the art. It should be understood that the present invention may be embodied in different ways and is not limited to the following embodiments. In the drawings, portions irrelevant to the description will be omitted for clarity. Like components will be denoted by like reference numerals throughout the specification

As used herein, the singular forms, “a”, “an” and “the,” are intended to include the plural forms as well, unless the context clearly indicates otherwise.

It will be understood that, although the terms “first”, “second”, and the like may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a “first” element or component discussed below could also be termed a “second” element or component, or vice versa, without departing from the scope of the present invention.

In the flowchart described with reference to the drawings, the order of operations may be changed, several operations may be merged, certain operations may be divided, and a certain operation may not be performed.

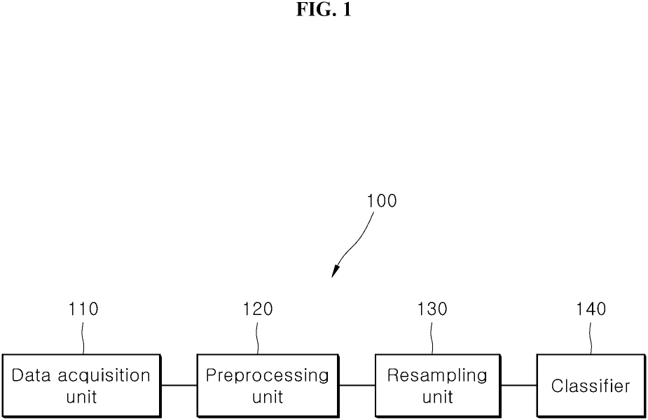

FIG. 1 is a diagram of one example of a heartbeat classification apparatus according to one embodiment of the present invention.

Referring to FIG. 1, a heartbeat classification apparatus 100 according to one embodiment is a deep learning-based heartbeat classification apparatus and is a computing device configured to classify heartbeats through machine learning with respect to a neural network (machine learning model) for carrying out an objective task or through a trained neural network. The objective task may include a task for heartbeat classification. To this end, the heartbeat classification apparatus 100 may use a heartbeat classification model as the machine learning model.

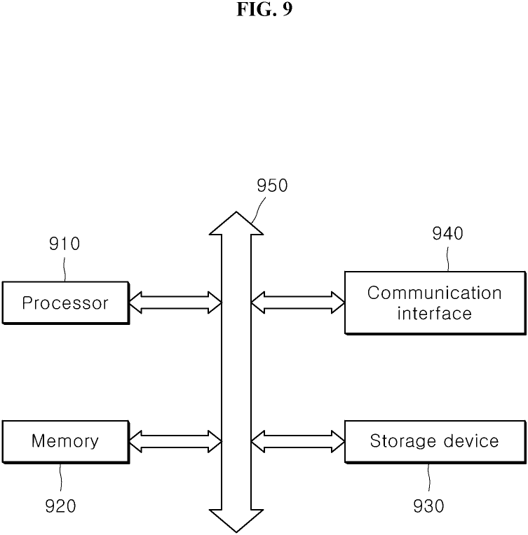

In some embodiments, the computing device may include any kind of devices having a computing function, which include a notebook computer, a desktop computer, a laptop computer, and a server, without being limited thereto. One example of the computing device will be described below with reference to FIG. 9.

Although FIG. 1 shows one example in which the heartbeat classification apparatus 100 is realized by a single computing device, it should be noted that functions of the heartbeat classification apparatus 100 may be realized by one or more computing devices.

The heartbeat classification apparatus 100 includes a classifier 140. In some embodiments, the heartbeat classification apparatus 100 may train a heartbeat classification model based on samples of a training data set. The samples are generated from electrocardiogram signals and are tagged with corresponding labels (kind of heartbeat). The heartbeat classification apparatus 100 may classify and predict classes by inputting the samples to the heartbeat classification model, may calculate loss based on the predicted classes and labels, and may train the heartbeat classification model by updating parameters of the heartbeat classification model through backpropagation of the loss. In some embodiments, the heartbeat classification apparatus 100 may input the samples, which correspond to the electrocardiogram signals, to the heartbeat classification model to classify the classes of the samples.

In some embodiments, the heartbeat classification apparatus 100 operated as a training apparatus may further include a resampling unit 130. Multiple samples included in the training data set may be classified into multiple class clusters according to classes to which the labels of the samples pertain. Among the multiple class clusters, a cluster having the largest number of samples corresponds to a majority class cluster and a cluster having the smallest number of samples corresponds to a minority class cluster. Hereinafter, a sample pertaining to the majority class cluster is referred to as a majority class sample and a sample pertaining to the minority class cluster is referred to as a minority class sample. The resampling unit 130 may convert a majority class sample selected from the majority class cluster into a minority class sample to regenerate the training data set according to a predetermined criterion.

In some embodiments, the heartbeat classification apparatus 100 may further include a data acquisition unit 110 and a preprocessing unit 120.

The data acquisition unit 110 may acquire an electrocardiogram signal for the data set of the heartbeat classification model. In some embodiments, the data acquisition unit 110 may select a database for training or verification of the heartbeat classification model among various databases. For example, the data acquisition unit 110 may select a MIT-BIH electrocardiogram database.

The preprocessing unit 120 may generate samples for the data set by preprocessing data obtained by the data acquisition unit 110. Alternatively, the preprocessing unit 120 may generate the samples for the data set through preprocessing of the electrocardiogram signal as a classification target. In some embodiments, the preprocessing unit 120 may remove various types of noise from data to extract heartbeat segments through preprocessing of the data.

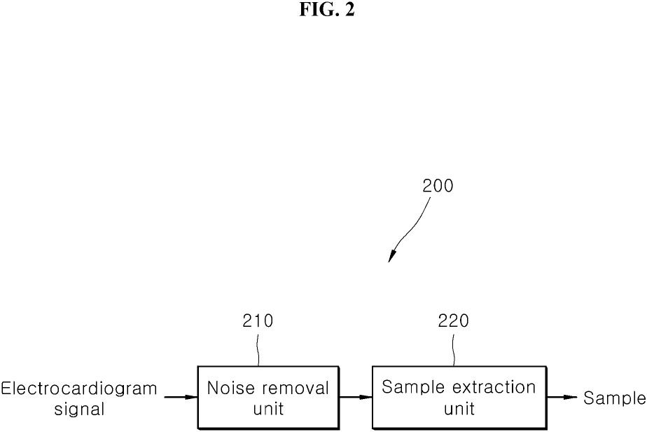

FIG. 2 is a diagram of one example of the preprocessing unit of the heartbeat classification apparatus according to the embodiment of the present invention.

Referring to FIG. 2, the preprocessing unit 200 includes a noise removal unit 210 and a sample extraction unit 220.

In general, the electrocardiogram signal can be damaged by various types of noise, such as motion artifacts and electric line interference. Such noise can make it difficult to extract desired information, thereby causing misclassification. Thus, the noise removal unit 210 removes noise from an electrocardiogram signal. In some embodiments, the noise removal unit 210 may remove baseline drifts and noise from the electrocardiogram signal.

In some embodiments, the noise removal unit 210 may include at least one filter for removal of the baseline drifts. In some embodiments, at least one filter may include two median filters. In the noise removal unit 210, a first median filter having a certain sliding window (for example, 200 ms sliding window) is applied to the electrocardiogram signal to remove a P wave and a QRS complex from the electrocardiogram signal, and a second median filter having a certain sliding window (for example, 500 ms sliding window) is applied to an output from the first median filter to remove a T wave therefrom. As a result, an output of the second median filter may include a baseline of the electrocardiogram signal. Accordingly, the noise removal unit 210 may remove the baseline drift from the electrocardiogram signal by subtracting an output of the second median filter from the electrocardiogram signal.

In some embodiments, the noise removal unit 210 may include a low-pass filter to remove noise. The noise removal unit 210 may remove high-frequency noise and power line noise by applying the low-pass filter to the electrocardiogram signal, from which the baseline drift has been removed. For example, the noise removal unit 210 may employ a low-pass filter having an order of 12 and a cutoff frequency of 35 Hz.

The sample extraction unit 220 extracts a desired sample (for example, segment) from the electrocardiogram signal, from which noise has been removed. In some embodiments, the sample extraction unit 220 may perform electrocardiogram peak detection to extract a desired segment. The sample extraction unit 220 may use, for example, Pan-Tompkins algorithm as a peak detection algorithm. Based on the peak detection algorithm, the sample extraction unit 220 may estimate T peaks after finding R peaks in the electrocardiogram signal and may extract a segment between consecutive T peaks as a sample. The sample extracted by the sample extraction unit 220 may be used as a sample of the training data set or a verification data set in a classifier (for example, classifier 140 of FIG. 1). In some embodiments, the sample may be generated in the form of a two-dimensional image.

In some embodiments, the sample extraction unit 220 may extract an RR interval from the electrocardiogram signal. The RR interval refers to an interval between two consecutive R waves in the electrocardiogram signal, for example, an interval between peak points of the two consecutive R waves.

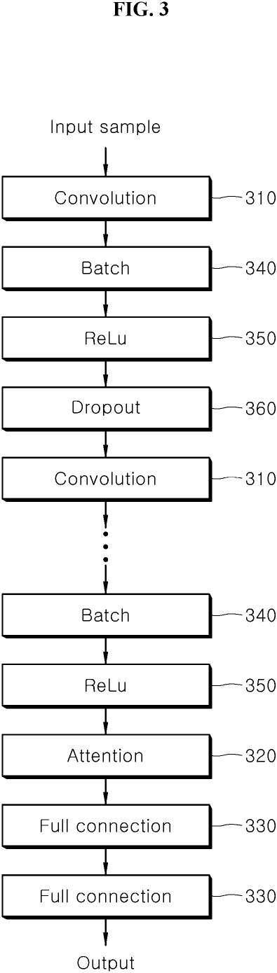

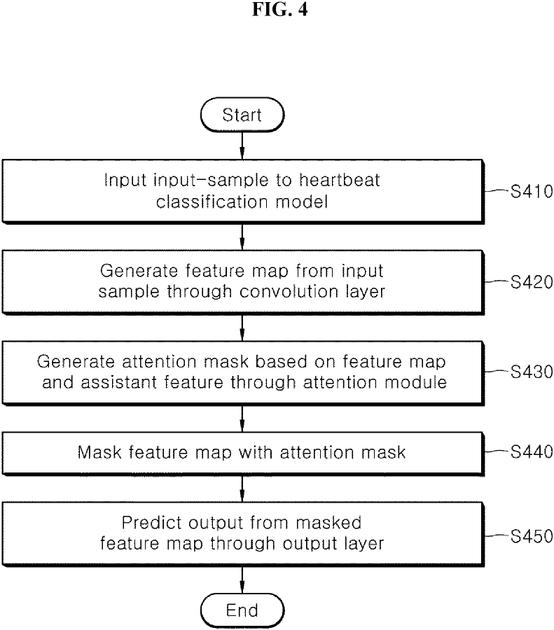

FIG. 3 is a diagram of one example of the classifier of the heartbeat classification apparatus according to the embodiment of the present invention, FIG. 4 is a flowchart illustrating one example of a heartbeat classification method or a training method of the heartbeat classification apparatus according to the embodiment of the present invention.

Referring to FIG. 3, the machine learning model of the classifier, that is, the heartbeat classification model 300, includes multiple convolution layers 310, an attention module 320 and an output layer 330, and receives an input sample corresponding to the electrocardiogram signal to output a class prediction value through classification of the input sample. The class prediction value may indicate a heartbeat of the electrocardiogram signal corresponding to the input sample. In some embodiments, the heartbeat may be, for example, an N beat (normal beat), a V beat (ventricular ectopic beat), an S beat (supraventricular ectopic beat), an F beat (fusion beat), or a Q beat (unknown beat).

Among the multiple convolution layers 310, for example, a first convolution layer 310 may receive an input sample from the preprocessing unit and may extract features from the input sample. Such features may be represented in the form of a feature map. Other convolution layers 310 may extract features again from features delivered from a preceding layer and may deliver the extracted features to the next layer. The convolution layers 310 may extract the features by applying a convolution filter to input data or input features. The convolution filter may extract the features by performing convolution operation on a receptive region of the input data or the input features. For example, a convolutional filter having a filter size of 16 with padding may be used.

In some embodiments, the heartbeat classification model 300 may further include a normalization layer 340 and an activation layer 350 disposed behind each of the convolution layers 310. In some embodiments, the normalization layer 340 may use a batch normalization function and the activation layer 350 may use a rectified linear unit (ReLu) function.

In some embodiments, the heartbeat classification model 300 may further include a dropout layer 360 behind the activation layer 350. For example, the dropout layer 360 may have a dropout value set to 0.5. In some embodiments, in use of the dropout layer 360, the dropout layer may not be present between the last convolution layer 310 and the attention module 320.

The attention module 320 may extract a target specific feature using an attention mask. The attention module 320 receives a feature map output (extracted) from a preceding layer and an assistant feature of a corresponding sample. In some embodiments, the assistant feature may be an RR interval of the sample, that is, of the electrocardiogram signal. The attention module 320 generates the attention mask based on the extracted feature map and the assistant feature, and outputs a masked feature map through application of the attention mask to the extracted feature map. As such, the target specific feature may be extracted through application of the attention mask to the feature map.

The output layer 330 may include a fully-connected layer (or dense layer) 330. The fully-connected layer 330 outputs a prediction value through classification from features (masked features) output from the attention module 320. The fully-connected layer may provide probability with respect to each label. In some embodiments, the output layer 330 may include multiple fully-connected layers 330. In some embodiments, the heartbeat classification model 300 may further include an activation layer behind the fully-connected layer 330. In some embodiments, the activation layer may use a sigmoid function.

Referring to FIG. 3 and FIG. 4, the heartbeat classification apparatus may input an input sample to the heartbeat classification model 300 (S410). The heartbeat classification apparatus may generate a feature map from the input sample through the multiple convolution layers 310 of the heartbeat classification model 300 (S420). The heartbeat classification apparatus generates an attention mask based on the feature map and an assistant feature through the attention module 320 of the heartbeat classification model 300 (S430), and masks the feature map with the attention mask (S440). The heartbeat classification apparatus performs classification of the input sample from the masked feature map through the output layer 330 of the heartbeat classification model 300 (S450). Through this process, the heartbeat classification apparatus may classify (predict) heartbeats corresponding to the electrocardiogram signal of the input sample from the input sample using the trained heartbeat classification model.

In some embodiments, when the heartbeat classification apparatus trains the heartbeat classification model, the heartbeat classification apparatus may repeatedly train the heartbeat classification model by inputting each of training samples of a training data set to the heartbeat classification model. In this case, each of the training samples is tagged with a label corresponding to correct answer classification of the corresponding electrocardiogram signal. The label may indicate a corresponding class among various classes of heartbeats. In this case, the heartbeat classification apparatus may calculate loss based on a predicted output and the label of the corresponding training model by inputting each of training models to the heartbeat classification model and may update parameters of the heartbeat classification model through backpropagation of the loss. The heartbeat classification model may be trained by repeating this process.

According to the embodiment described above, the heartbeat classification model may be trained based on the target specific feature through the attention module and arrhythmias can be detected by classifying heartbeats using the trained heartbeat classification model.

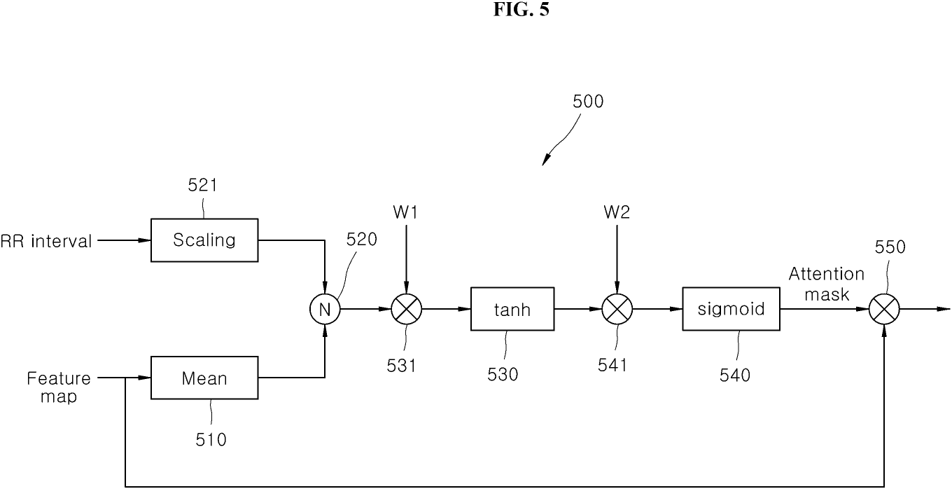

FIG. 5 is a diagram of one example of the attention module of the heartbeat classification apparatus according to the embodiment of the present invention.

Referring to FIG. 5, the attention module 500 includes a statistical value calculation unit 510, a normalization unit 520, a neural network, and a mask application unit 550.

The statistical value calculation unit 510 receives a feature map extracted from the preceding layer and calculates a statistical value of the feature map. In some embodiments, the statistical value may be a mean value and will now be described as the mean value. In some embodiments, when the feature map has a size of K × L, the statistical value calculation unit 510 may calculate a mean value of features in each column and may output a mean value of 1 × L. For example, the statistical value calculation unit 510 may calculate a mean value in each column, as in Equation 1:

$${\text{Feature}}_{m e a n} = \frac{1}{K} {\sum }_{k = 1}^{K} {f}_{c , k}$$

where ƒ_{c,k} is a value of the k^{th} feature in each column of the feature map and K is the number of features included in each column of the feature map.

The normalization unit 520 normalizes the mean value of the feature map with the assistant feature. In some embodiments, the assistant feature may be an RR interval of the sample corresponding to the feature map. In some embodiments, the normalization unit 520 may scale the assistant feature (521) before normalization of the mean value of the feature map with the assistant feature. For example, the normalization unit 520 may normalize the mean value of the feature map with the assistant feature, as in Equation 2:

$$T o t a {l}_{s t a t} = \frac{F e a t u r {e}_{m e a n}}{α ∗ R {R}_{i}}$$

where RRᵢ is an RR interval and α is a scaling coefficient.

The neural network generates the attention mask from the mean value of the normalized feature map and may include one or more activation layers 530, 540. An output of the normalization unit 520 may be input to the activation layer 530 after multiplying the output by a weight value W1 and shifting the output by a predetermined value b1 (531). In addition, an output of the normalization unit 530 may be input to the activation layer 540 after multiplying the output by a weight value W2 and shifting the output by a predetermined value b2 (541). In some embodiments, the activation layer 530 may use the tanh function as the activation function and the activation layer 540 may use the sigmoid function as the activation function. In this case, the output Vs of the activation layer 531 and the output A_{c,k} of the activation layer 532 may be obtained according to Equations 3 and 4.

$${V}_{s} = \text{tanh} \left(T o t a {l}_{s t a t} ∗ W 1 + b 1\right)$$

$${A}_{c , k} = \text{sigmoid} \left({V}_{s} ∗ W 2 + b 2\right)$$

The output of the activation layer 532 corresponds to the attention mask A_{c,k} and the mask application unit 540 outputs a masked feature through application of the attention mask A_{c,k} to the feature map. In some embodiments, the mask application unit 550 may output the masked feature through multiplication of the feature map ƒ_{c,k} by the attention mask A_{c,k}, as in Equation 5. In some embodiments, since the attention mask has a size of 1 × L, all features in each column of the feature map may be multiplied by the same attention mask. The mask value reflected in the features may reflect importance of the corresponding class.

$${F}_{m a s k e d} = {f}_{c , k} ∗ {A}_{c , k}$$

The assistant feature described above may be used to extract features having the highest relevancy through combination of information related to a morphological pattern. Accordingly, the heartbeat classification apparatus may improve distinction between different classes by extracting a target specific feature using the assistant feature.

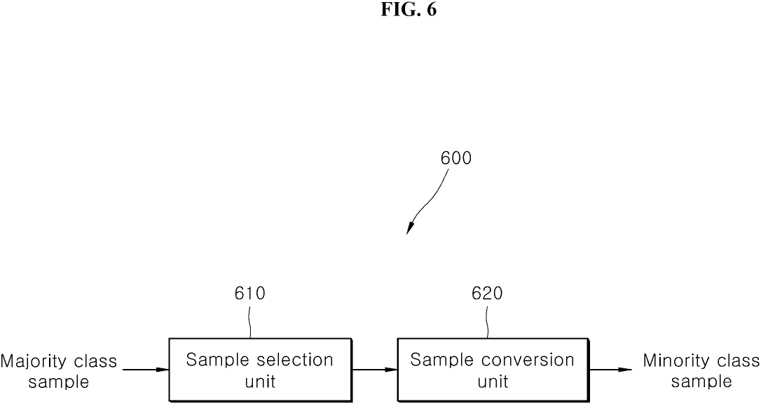

Next, the resampling unit of the heartbeat classification apparatus will be described with reference to FIG. 6 to FIG. 8.

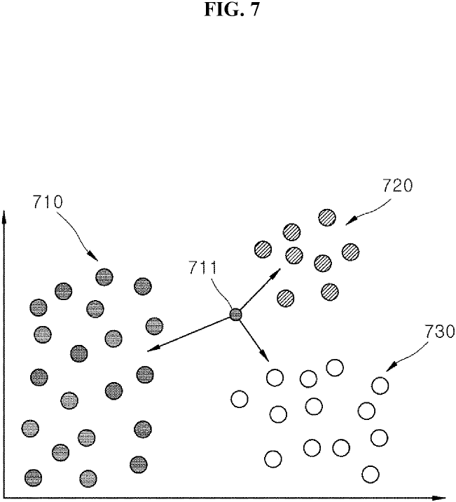

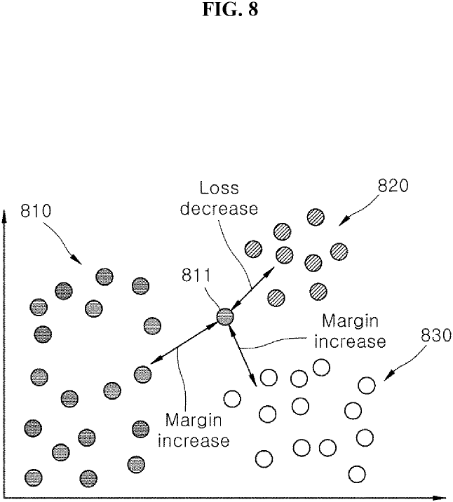

FIG. 6 is a diagram of one example of the resampling unit of the heartbeat classification apparatus according to the embodiment of the present invention. FIG. 7 is a view illustrating a sample suitable for conversion in the heartbeat classification apparatus according to the embodiment of the present invention, FIG. 8 is a view illustrating conversion of a sample in the heartbeat classification apparatus according to the embodiment of the present invention.

A resampling unit 600 includes a sample selection unit 610 and a sample conversion unit 620.

The multiple samples included in the training data set may be classified into the multiple class clusters according to the classes to which the labels of the samples pertain. Among the multiple class clusters, a cluster having the largest number of samples corresponds to the majority class cluster and a cluster having the smallest number of samples corresponds to the minority class cluster.

The sample selection unit 610 may select a suitable sample among the majority class samples according to a predetermined criterion. In some embodiments, the predetermined criterion may include similarity to minority class samples. As shown in FIG. 7, among the majority class samples 710, the sample selection unit 610 may select a sample 711 having higher similarity to the minority class samples 720 than other class samples 710, 730.

In some embodiments, similarity between the samples may be determined based on the Euclid distance. The sample selection unit 610 may select a certain sample x_{maj,k} among the majority class samples and may calculate a distance between the selected sample x_{maj,k} and a sample x_{min,c} corresponding to a center of the minority class samples. In addition, the sample selection unit 610 may calculate a distance between the selected sample x_{maj,k} and a sample x_{all,c} corresponding to a center of samples in each class excluding the minority class samples. When the distance between the selected sample x_{maj,k} and the sample x_{min,c} corresponding to the center of the minority class samples is shorter than the distances between the selected sample x_{maj,k} and the samples x_{all,c} corresponding to the center of all of other class samples. The sample selection unit 610 may select the corresponding sample x_{maj,k} as a suitable sample according to the predetermined criterion. For example, the sample selection unit 610 may select a majority class sample x_{maj,k} satisfying Equation 6 as the suitable sample.

$$d \left({x}_{m i n , c} , {x}_{m a j , k}\right) < d \left({x}_{a l , c} , {x}_{m a j , k}\right)$$

where d() represents the Euclid distance.

The sample conversion unit 620 converts the sample, which is selected by the sample selection unit 610, into a minority class sample. The sample conversion unit 620 tags the converted sample with the label of a source class. In some embodiments, the sample conversion unit 620 may perform sample conversion in an optimized way. In the optimized way, a target function may be optimized such that a converted (generated) sample provides higher classification performance in a target class. In some embodiments, the sample conversion unit 620 may convert the selected sample into a source class sample so as to improve sample similarity to a target minority class while reducing similarity to other classes. In some embodiments, as shown in FIG. 8, the sample conversion unit 620 may increase margins between a selected sample 811 and boundaries of other classes 810, 830 based on a constant factor in order to improve reliability of the classifier for classification of the minority class sample. In addition, as shown in FIG. 7, the sample conversion unit 620 may perform optimization so as to reduce loss with respect to a minority class 820.

For example, the sample conversion unit 620 may convert the sample x_{maj,k} into the minority class sample, as shown in Equation 7:

$${x}^{∗} = a r g m i n \text{d} \left({x}_{m i n , c} , {x}_{m a j , k}\right) - \left(\text{β} + \text{d} \left({x}_{a l , c} , {x}_{m a j , k}\right)\right)$$

where β is a constant factor for margin induction.

The sample generated by the sample conversion unit 620 may be input to the heartbeat classification model after being tagged with the label of the target class (source class).

Since the minority class has a very small number of samples, regeneration of a new sample from a limited number of minority class samples can cause unsuitable classification and classification failure together with reduction in generalization capability of the heartbeat classification model. However, the resampling unit described above generates the minority class sample from the majority class sample, thereby improving generalization capability of the heartbeat classification model without unbalanced class data.

Next, one example of the computing device capable of realizing the heartbeat classification apparatus, the heartbeat classification method or the training method according to the embodiment of the present invention will be described with reference to FIG. 9.

FIG. 9 is a block diagram of one example of a computing device according to one embodiment of the present invention.

Referring to FIG. 9, the computing device includes a processor 910, a memory 920, a storage device 930, a communication interface 940, and a bus 950. The computing device 900 may further other components generally used in the art.

The processor 910 controls overall operation of each of components constituting the computing device 900. The processor 910 may be implemented by at least one of various processing units, such as a central processing unit (CPU), a microprocessor unit (MPU), a microcontroller unit (MCU), a graphics processing unit (GPU), and the like, and may also be implemented by a parallel processing unit. Further, the processor 910 may perform operation of a program for implementation of the heartbeat classification method or the training method described above.

The memory 920 stores various data, commands and/or information. The memory 920 may load a computer program from the storage device 930 in order to execute the heartbeat classification method or the training method. The storage device may nontemporarily store the program. The storage device 930 may be implemented by a non-volatile memory.

The communication interface 940 supports wired and wireless Internet communication of the computing device 900. In addition, the communication interface 940 may support various communication methods as well as Internet communication.

The bus 950 provides a communication function between components of the computing device 900. The bus 950 may be implemented by various types of buses, such as an address bus, a data bus, and a control bus.

The computer program may include instructions that allow the processor 910 to perform the heartbeat classification method or the training method when loaded into the memory 920. That is, the processor 910 may perform operation for the heartbeat classification method or the training method by executing the instructions.

In some embodiments, for implementation of the heartbeat classification method, the computer program may include instructions that perform inputting of a sample generated from an electrocardiogram signal to a heartbeat classification model, generation of a feature map from the sample through multiple first layers of the heartbeat classification model, generation of an attention mask based on an assistant feature generated from the feature map and the sample, generation of a masked feature map by masking the feature map with the attention mask; and classification of the sample from the masked feature map through a second layer of the heartbeat classification model.

In some embodiments, for implementation of the method of training a heartbeat classification model, the computer program may include instructions that perform, upon classification of multiple samples generated from an electrocardiogram signal into multiple class clusters according to classes to which labels of the samples pertain, selection of a first sample from a majority class cluster having the largest number of samples among the multiple class clusters based on a predetermined criterion; conversion of the first sample into a second sample pertaining to a source class cluster having the smallest number of samples among the multiple class clusters; generation of a data set from the multiple samples and the second sample; and training of the heartbeat classification model by inputting an input sample selected from the data set to the heartbeat classification model.

The heartbeat classification method or the training method according to the embodiment of the present invention may be implemented by a computer-readable computer program on a computer-readable medium. In one embodiment, the computer-readable medium may be a movable recording medium or a stationary recording medium. In another embodiment, a computer program recorded on the computer-readable medium may be transferred to another computing device through a network including the Internet to be installed in and executed by the other computing device.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Although some embodiments have been described with reference to the drawings, it should be understood that the present invention is not limited to these embodiments, and that various modifications, changes, and alterations can be made without departing from the spirit and scope of the invention. Therefore, the scope of the invention should be limited only by the accompanying claims and equivalents thereof.

1. A heartbeat classification method carried out by a computing device, the heartbeat classification method comprising the steps of:
inputting a sample generated from an electrocardiogram signal to a heartbeat classification model;
generating a feature map from the sample through multiple first layers of the heartbeat classification model;
generating an attention mask based on an assistant feature generated from the feature map and the sample;
generating a masked feature map by masking the feature map with the attention mask; and
performing classification of the sample from the masked feature map through a second layer of the heartbeat classification model.

2. The heartbeat classification method according to claim 1, wherein the assistant feature comprises an RR interval of the electrocardiogram signal corresponding to the sample.

3. The heartbeat classification method according to claim 1, wherein the step of generating an attention mask comprises:
calculating a statistical value of the feature map;
generating a normalized statistical value through normalization of the statistical value with the assistant feature; and
generating the attention mask from the normalized statistical value through at least one activation layer.

4. The heartbeat classification method according to claim 3, wherein the step of generating an attention mask further comprises inputting the normalized statistical value to the activation layer after multiplying the normalized statistical value by a weight value and shifting the normalized statistical value by a predetermined value.

5. The heartbeat classification method according to claim 1, wherein the multiple first layers comprise multiple convolution layers and the second layer comprises at least one fully-connected layer.

6. A method of training a heartbeat classification model carried out by a computing device, comprising the steps of: upon classification of multiple samples generated from an electrocardiogram signal into multiple class clusters according to classes to which labels of the samples pertain,
selecting a first sample from a majority class cluster having the largest number of samples among the multiple class clusters based on a predetermined criterion;
converting the first sample into a second sample pertaining to a source class cluster having the smallest number of samples among the multiple class clusters;
generating a data set from the multiple samples and the second sample; and
training the heartbeat classification model by inputting an input sample selected from the data set to the heartbeat classification model.

7. The method according to claim 6, wherein the predetermined criterion comprises similarity to a sample pertaining to the source class cluster.

8. The method according to claim 6, wherein the step of selecting a first sample comprises:
calculating a first distance from the first sample to a sample corresponding to a center of the source class cluster;
calculating a second distance from the first sample to a sample corresponding to a center of each of remaining class clusters excluding the source class cluster among the multiple class clusters; and
selecting the first sample when the first distance is smaller than the second distance.

9. The method according to claim 6, wherein the step of converting the first sample into the second sample comprises tagging the second sample with a label of the source class cluster.

10. The method according to claim 6, wherein the step of converting the first sample into the second sample comprises increasing margins between the second sample and boundaries of the remaining class clusters based on a constant factor.

11. The method according to claim 6, wherein the step of training the heartbeat classification model comprises:
generating a feature map from the input sample through multiple first layers of the heartbeat classification model;
generating an attention mask based on an assistant feature generated from the feature map and the input sample;
generating a masked feature map by masking the feature map with the attention mask; and
performing classification of the input sample from the masked feature map through a second layer of the heartbeat classification model.

12. The method according to claim 11, wherein the assistant feature comprises an RR interval of the electrocardiogram signal corresponding to the input sample.

13. The method according to claim 11, wherein the step of generating an attention mask comprises:
calculating a statistical value of the feature map;
generating a normalized statistical value through normalization of the statistical value with the assistant feature; and
generating the attention mask from the normalized statistical value through at least one activation layer.

14. The method according to claim 13, wherein the step of generating an attention mask further comprises inputting the normalized statistical value to the activation layer after multiplying the normalized statistical value by a weight value and shifting the normalized statistical value by a predetermined value.

15. The method according to claim 11, wherein the multiple first layers comprise multiple convolution layers and the second layer comprises at least one fully-connected layer.

16. A heartbeat classification apparatus comprising:
a memory storing at least one instruction; and
a processor,
wherein the processor executes the instruction to input a sample generated from an electrocardiogram signal to a heartbeat classification model, to generate a feature map from the sample through multiple first layers of the heartbeat classification model, to generate an attention mask based on an assistant feature generated from the feature map and the sample, to generate a masked feature map by masking the feature map with the attention mask; and to perform classification of the sample from the masked feature map through a second layer of the heartbeat classification model.

17. The heartbeat classification apparatus according to claim 16, wherein the assistant feature comprises an RR interval of the electrocardiogram signal corresponding to the sample.

18. The heartbeat classification apparatus according to claim 16, wherein the processor calculates a statistical value of the feature map, generates a normalized statistical value through normalization of the statistical value with the assistant feature; and generates the attention mask from the normalized statistical value through at least one activation layer.

19. The heartbeat classification apparatus according to claim 18, wherein the processor inputs the normalized statistical value to the activation layer after multiplying the normalized statistical value by a weight value and shifting the normalized statistical value by a predetermined value.

20. The heartbeat classification apparatus according to claim 16, wherein the multiple first layers comprise multiple convolution layers and the second layer comprises at least one fully-connected layer.
